(19) United States
(12) Patent Application Publication
(10) Pub. No.: US 2004/0101870 A1
(43) Pub. Date: May 27, 2004

(54) Microvolume biochemical reaction chamber

(76) Inventors: Bruno S. Caubet, Le Blanc Mesnil (FR); Thierry DeLumley, Paris (FR); Olivier Lobet, Fontainebleau (FR); Uwe R. Muller, Waukegan, IL (US); Jean-Pierre Themont, Montigny Sur Loing (FR)

Correspondence Address: CORNING INCORPORATED, SP-TI-3-1, CORNING, NY, 14831

(21) Appl. No.: 10/385,083

(22) Filed: Mar. 10, 2003

(30) Foreign Application Priority Data
Nov. 26, 2002 (EP) EP 02 292917.8

(51) Int. Cl. C12M 1/34

(52) U.S. Cl. 435/6; 435/287.2; 435/288.3

(57) ABSTRACT

Methods and apparatus for performing biomolecular reactions using microvolumes of reagents are disclosed. The apparatus and methods include a chamber having a height less than 50 microns and means for mixing the extremely small volume of fluid in the chamber. The decreased volumes combined with mixing greatly improved microarray hybridization signal strength.

### CROSS-REFERENCE TO RELATED APPLICATIONS

This application claims the benefit of European Patent Application No. 02 292 917.8, filed Nov. 26, 2002, entitled MICROVOLUME BIOCHEMICAL REACTION CHAMBER, the content of which is relied upon and incorporated herein by reference in its entirety.

1. Field of the Invention

This invention relates to reaction chambers used for biomolecular and biochemical analysis. More particularly, the invention relates to methods and apparatus that use small volumes of reagent for performing biochemical reactions.

2. Background of the Invention

High density molecular arrays are solid surfaces containing surface bound biomolecules arrayed in specific positions and used in analysis of solutions containing a mixture of analytes. In some types of arrays, such as arrays used in hybridization experiments, the surface bound biomolecules are called probe molecules and the mixture of analytes contains what are sometimes called target molecules. Examples of such biomolecules include but are not limited to proteins, antibodies, oligonucleotides, nucleic acids, peptides and polypeptides. For example, DNA microarrays are used to identify which genes are “turned on or off” in a cell or tissue, and to evaluate the extent of a gene presence under various conditions. After hybridization with cDNA labeled with a fluorochrome, the slides can be read with a fluorescence scanning device. Presence of a specific gene in the sample is revealed by fluorescence of the corresponding hybridized spot on the chip. Fluorescence intensity is related to the number of hybridized strands at the spot, which is related to the gene abundance in the sample. Each spot location provides an address for later reference to each spot of nucleic acid.

Hybridization techniques utilize markers such as radioactive or fluorescent compounds to label particular nucleic acid sequences that are complementary to the nucleic acid sequences on a substrate such as a glass slide. Signal measurement equipment is then utilized to measure each address on the array to determine if the labeled sequences have attached to the complementary or partially complementary sequence on the glass slide. The resulting slide is examined using an evaluation procedure such as, for example, microscopy, autoradiography, fluorescence measurement, photon emission, or the like. A single hybridization procedure may involve as many as thirty or more controlled step sequences.

Biochemical reactions such as hybridization reactions require adequate interaction between the target molecules in the fluid and the probe molecules bound to the substrate. One typical arrangement for performing hybridization reactions involves the use of an array of probe molecules immobilized on a substrate having a surface area typically less than a few square centimeters. Appropriate substrate materials include fused silica, glass, and silicon, however, glass slides treated with a chemical moiety to facilitate attachment of probes are preferred in the industry. After the fluid containing the target molecules is placed in contact with a substrate, a second glass slide or cover slip is used to cover the fluid. Hereinafter, this technique will be referred to as the cover slip technique. Hybridization reactions using the cover slip technique typically take up to several hours.

It is important to control the reaction conditions to improve the reliability and reproducibility of biochemical reactions. Reducing the volume of the chamber increases the concentration of reactants of the chamber, and in turn increases the sensitivity of the assay. However, merely placing a second slide over a first slide containing an array of surface bound biomolecules and a fluid containing analyte molecules does not adequately control the volume of fluid across the surface area of the slide. In addition, it is difficult to ensure precise mixing of the fluid between the two slides. Furthermore, fluid has a tendency to leak out from between the two slides during use. Although it is possible to contain the fluid by sealing the edges of the two slides with an adhesive, this approach is time consuming and can introduce contaminants into the fluid. Another approach to containing the fluids involves the use of an O-ring or gasket between the substrate and the cover slide. A limitation to this approach is that such O-rings and gaskets are typically greater than 1.5 mm thick, which provides a very large space between the slides. One drawback of thicker spaced biochemical reaction chambers is that they require large quantities of fluid.

A typical thickness of a solution layer between two slides is between 50 and 300 microns. Reaction of surface bound biomolecules and analytes in solution between two slides is also limited by poor mixing, particularly when the thickness of the reaction chamber is less than 100 microns. Poor mixing leads to solutions that are not homogeneous, and poor mixing can cause poor reaction kinetics, low efficiency, low sensitivity and low yield. When mixing is poor, diffusion controls the movement of reactants or analytes in the fluid to the substrate surface. Therefore, reaction of surface bound biomolecules and analytes contained in solution is a diffusion-limited process that depends on the size of the molecules and the diffusion distance from the bulk to the surface. Oligomers of different sequences, length and concentration will have different hybridization rate. After a sufficient period of time (e.g., 14 hours) all oligomers at the same concentration should reach the same plateau and surface coverage. The efficiency of a diffusion-limited interaction of a solid bound probe and an analyte in solution is never more than a few percent. In addition, in hybridization procedures utilizing a cover slip over a slide containing an array of probe molecules and a solution of target molecules, if the cover slide is not exactly parallel to the substrate surface containing the probe molecules, there may be more target molecules present in the sections of the array where the liquid is thicker than in the thinner sections.

Some hybridization chambers have been developed to provide a significant fluid movement and proper mixing. However, this has been done at the expense of larger volumes and leads to probe dilution. In commercial hybridization stations, volumes used typically range from 150 to 300 uL. By providing mixing, gains in sensitivity have been measured at about 3-fold compared to the cover slip and substrate approach.

U.S. Pat. No. 6,258,593 discloses an apparatus that includes a substrate having a planar surface, a plastic cover having a peripheral lip which sealingly contacts the substrate to form a reaction chamber, and a fastener for forming seal between the cover and the substrate. The chamber height ranges from 50 to 500 microns. The patent further discloses that mixing of solutions in the reaction chamber is facilitated by inclusion of surfactant solution in the reaction chamber.

U.S. Pat. No. 5,922,604 discloses a reaction chamber that includes a space enclosed between two opposing walls having at least two heights to cause capillary action between the sections of the chamber having different heights. The '604 patent claims that this design aids in movement, facilitates removal of fluid from the chamber and is an improvement over the use of an air stream to force or blow fluid out of the chamber.

U.S. Pat. No. 5,910,288 discloses a reaction chamber for mixing thin films of fluids that does not require removal of fluid from the reaction chamber for mixing in separate chambers external to the reaction chamber. The reaction chamber includes two surfaces having opposing inner faces. Fluid in the reaction chamber in U.S. Pat. No. 5,910,288 by moving the inner face of one of the surfaces by shearing, compressing, rotating or inducing tension to one of the surfaces. While the mixing method disclosed in the '288 patent purportedly has certain advantages, the application of force to one of the surfaces can be problematic because force on the surface of the slide can lead to breakage. In addition, shear, compression and rotational forces may adversely affect biological samples contained in the chamber.

Although a variety of apparatus and methods exist related to reaction chambers which are used in biochemical assays, there still exists a need for improved apparatus and methods for performing such assays. In particular, there exists a need for apparatus and methods that provide adequate mixing of microvolumes of fluids contained in reaction chambers. In addition, apparatus and methods are needed that provide reproducible and controllable results with low signal to noise ratio of microarrays when read with fluorescence scanning devices.

### SUMMARY OF INVENTION

One embodiment of present invention relates to a biochemical reaction device. This embodiment includes a generally planar base substrate having an inner surface including a specimen area having at least one biomolecule attached to the specimen area and a generally planar cover substrate having an inner surface opposing and substantially parallel to the inner surface of the base substrate. This embodiment further includes a peripheral spacer disposed between the inner surfaces of the base substrate and the cover substrate and forming a fluid tight seal between the cover substrate and the base substrate, and the inner surfaces of the base substrate and the peripheral spacer define a chamber having a height less than 50 microns and a fluid volume of less than 75 microliters. A pair of fluid ports extending through the cover substrate is disposed on opposite ends of the specimen area, and each of the ports is in fluid communication with reservoirs. This embodiment further includes means for moving fluid through the channels, the pair of fluid ports and the chamber. In certain embodiments, the reservoirs include fluid channels, the construction of which will be described in more detail below. In certain embodiments, the channels are disposed between a third generally planar substrate and an upper surface of the cover substrate and the direction of the fluid flow through the channels is generally parallel to the inner surface of the cover substrate.

In certain embodiments, the means for moving fluid includes a pump. The pump must be capable of pumping microvolumes of fluid less than 75 microliters, and in some embodiments less than 20 microliters through the reservoirs or channels, the fluid ports and the chamber. The pump can include a syringe pump or other similar type of pumping device. In other embodiments, particularly when the height of the chamber is less than about 30 microns, the means for moving fluid includes a vacuum and pressure pump in fluid communication with the ports. In other embodiments, the fluid movement means can include a pump driven by one or more piezoelectric elements.

In some embodiments, a sensor is disposed proximate at least one of the channels or reservoirs to monitor the volume of fluid in the channel or reservoir. In certain embodiments, the sensor is in communication with the means for moving the fluid, such as a source of pressure and vacuum, and the sensor is operative to control the pressure and vacuum to change the direction of fluid flow.

In certain embodiments, the peripheral spacer is made from a glass frit. In other embodiments, the peripheral spacer is made from a polymer, such as for example, a plastic film. The peripheral spacer can be made from other types of materials as well such as a liquid or gel material that can form a fluid tight seal such as grease. The peripheral spacer can be made from combinations of the above material as well. In still other embodiments, the height of the chamber is less than about 20 microns and the fluid volume of the chamber is less than about 30 microliters. Other embodiments of the invention relate to methods of performing a biochemical reactions such as hybridization assays that include the step of providing biochemical reaction devices of the type described above and including a microarray of biomolecules attached to an inner surface of the base substrate. The method further includes the step of moving fluid between the pair of reservoirs and their respective ports and through the chamber. In certain embodiments, the microarray includes biomolecules bound to the inner surface of the base substrate and the fluid contains analyte biomolecules. In certain embodiments, the fluid is moved through the pair of reservoirs by a pump such as a syringe pump connected to the pair of reservoirs. In other embodiments, the fluid movement is achieved by connecting a source of pressure and vacuum to the reservoirs. Alternatively the fluid movement may be accomplished by utilizing a pump including piezolectrically driven elements. According to some embodiments, the pressure may be provided by pressurized heated or gas.

In some embodiments, the method may further include monitoring the volume of fluid in at least one of the reservoirs. In certain embodiments, the pump may be controlled based on the volume of fluid in at least one of the reservoirs. In these embodiments, the pump may be connected to a sensor that monitors the volume of the fluid in at least one of the reservoirs. Both the pump and the sensor can be connected to a controller that controls the operation of the pump based on the volume of fluid in one of the reservoirs. In certain embodiments, the direction of fluid movement in the chamber based on the volume of fluid in at least one of the reservoirs is controlled by the controller. For example, in embodiments that use a pressure and vacuum fluid movement system, the controller can signal the pressure and vacuum apparatus to alternate between vacuum and pressure to alternate the direction of fluid movement in the system.

According to certain embodiments of the present invention, reducing the total volume of reaction chamber and the total thickness combined with providing fluid movement by pumping greatly improves the sensitivity of the fluorescence signal when compared with the conventional reaction method utilizing a cover slip placed directly over a solution on a base substrate. According to the present invention, volumes are reduced from about 150 microliters used in conventional reaction chambers to less than about 75 microliters, and in some embodiments, as low as about 20 microliters. The height of the reaction chamber or thickness of the fluid film is reduced from greater than about 50 microns to below about 50 microns, and in preferred embodiments, as low as about 10 microns. When combined with fluid movement by pumping using as syringe pump or pressure and a vacuum, applicants have observed up to about a 50 times gain in sensitivity in fluorescence measurements on substrates compared to traditional hybridization devices utilizing a cover slip placed directly over a solution placed on a base substrate.

Additional advantages of the invention will be set forth in the following detailed description. It is to be understood that both the foregoing general description and the following detailed description are exemplary and are intended to provide further explanation of the invention as claimed.

### BRIEF DESCRIPTION OF THE DRAWING

FIG. 1 is a side view of a biochemical reaction chamber device including a pump connected to one of the reservoirs according to one embodiment of the invention;

FIG. 2 is a top view of the biochemical reaction chamber device shown in FIG. 1;

FIG. 3 is an exploded perspective view of a biochemical reaction chamber device according to another embodiment of the invention;

FIG. 4 is a perspective view of the reaction chamber device shown in FIG. 3 in an assembled configuration;

FIG. 5 is a perspective view of the reaction chamber shown in FIG. 4 with the cover substrate removed;

FIG. 6 is a top view of the reaction chamber shown in FIG. 5;

FIG. 6A is a side cross-sectional view of the reaction chamber shown in FIG. 6 taken along line 6A;

FIG. 7 is a perspective view of a reaction chamber station in accordance with another embodiment of the invention;

FIG. 8 is a perspective view of a portion of a reaction chamber station in accordance with another embodiment of the invention;

FIG. 9 is a schematic representation of a microarray printout;

FIG. 10 is a graph of normalized relative fluorescent unit (RFU) versus the human genes printed on a slide with an undiluted target solution in biochemical reaction chambers having chamber heights of 50 microns and 100 microns with and without fluid movement;

FIG. 11 is a graph of normalized relative fluorescent unit (RFU) versus the human genes printed on a slide with a probe diluted 10 times in biochemical reaction chambers having chamber heights of 50 microns and 100 microns with and without fluid movement; and

FIG. 12 is a graph of normalized relative fluorescent unit (RFU) versus the human genes printed onto the slide with a probe diluted 50 times in biochemical reaction chambers having chambers heights of 50 microns and 10 microns with and without fluid movement.

### DETAILED DESCRIPTION

Before describing several exemplary embodiments of the invention, it is to be understood that the invention is not limited to the details of construction, process steps, reagents and biomolecules set forth in the following description. The invention is capable of other embodiments and of being practiced or being carried out in various ways.

The present invention relates to methods and devices for performing biochemical reactions and enhancing the reaction or interaction between surface bound biomolecules and analytes or biomolecules contained in solution. Surface bound biomolecules are often referred to as probes and the analytes or biomolecules contained in solution are often referred to as targets. One type of reaction in which surface bound probes and target molecules in solution interact are called hybridization reactions. As used herein, the term hybridization refers to binding between complementary or partially complementary molecules. The term probe means a molecule adhered to a substrate. The term target means a molecule in solution.

However, the present invention is not limited to any specific type of hybridization reaction, and the chambers and methods of the various embodiments of the present invention can be used in a wide variety of biochemical reactions. Examples of a few of the types of reactions that the present invention can be used to enhance, include, but are not limited to fluorescent in situ hybridization (FISH), protein array reactions, immunostaining applications and general staining or histochemical reactions. In FISH reactions, the analytes in solution include DNA probes (oligomers, cDNAs, PCR fragments, or clones such as plasmids, BACs (bacterial artificial chromosomes), PACs (phage artificial chromosomes), cosmids, or phage chromosomes, and the surface bound biomaterial (analyte binding partner) can include whole human chromosomes or fragments thereof, that are typically contained in human metaphase spreads, or where the affixed biomaterial is whole human cells or nuclei, or even extracted human DNA, where the DNA has been made available for hybridization to the analyte in solution. In protein arrays, the analyte in solution typically includes one or more antibodies or substrates that are labeled directly or indirectly, and the surface bound biomaterial includes one or more proteins that have affinity for one or more of the analytes in solution. In immunostaining reactions, the analyte in solution typically includes one or more antibodies that are labeled directly or indirectly, and the surface bound biomaterial includes one or more antigens of the type including DNA, RNA, protein, cell membranes, metabolites, whole cells, bacteria, fungi, viruses and the like. In other types of immunostaining reactions, the analyte in solution includes one or more antigens of the type including DNA, RNA, protein, cell membranes, metabolites, whole cells, bacteria, fungi, viruses and the like, and the surface bound biomaterial includes one or more antibodies. In general histochemical or general staining reactions, the surface bound biomaterial is any type of biomaterial and the analyte in solution includes one or more of commonly used stains, such as Eosin, Hematoxilyn, etc. Thus, it is to be understood that the devices and methods of the present invention can be used in a wide variety of biochemical reactions to overcome diffusion limitations imposed on the interaction between surface bound biomaterials or biomolecules and analytes contained in solution by reducing the volume of a reaction chamber, which increases the effective concentration, and physically moving the fluid in the chamber.

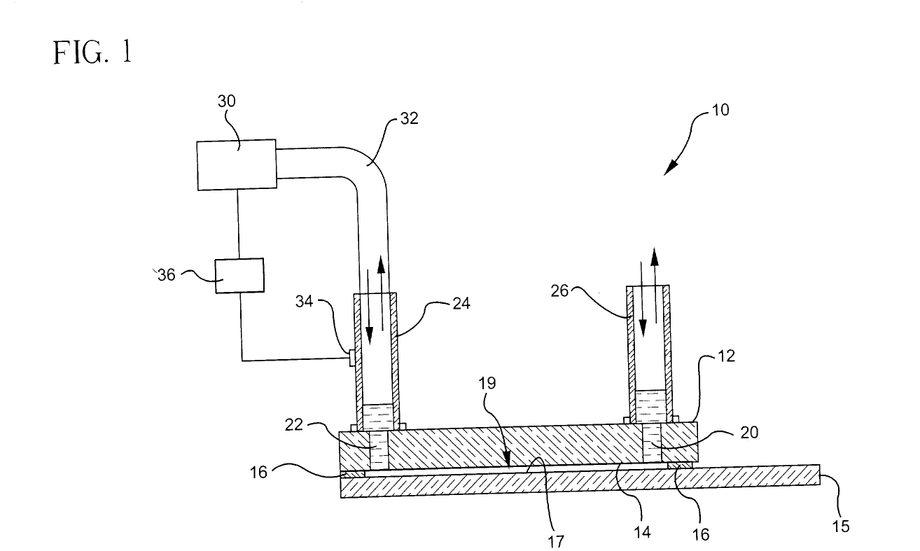

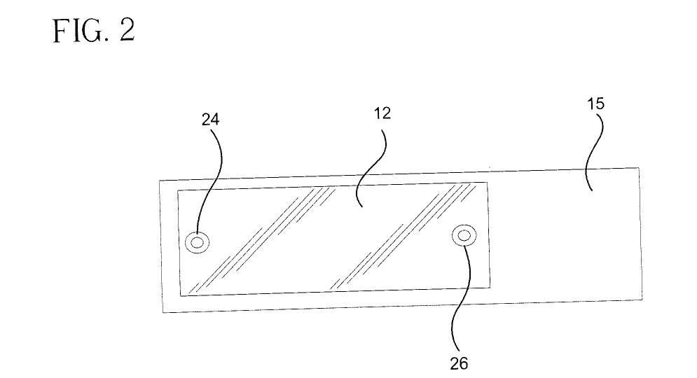

An exemplary embodiment of a reaction chamber device is shown in FIGS. 1 and 2 and designated generally as 10. The device 10 includes a generally planar cover substrate 12 having an inner surface 14. The cover substrate 12 is adapted to be assembled in a substantially parallel configuration to a generally planar base substrate 15 having an inner surface 17 including a specimen area having at least one biomolecule attached to the specimen area. A peripheral spacer 16 surrounds the periphery of the inner surface of the cover substrate 12, and when the cover substrate 12 is assembled to the base substrate 15, the peripheral spacer forms a fluid tight seal between the two substrates. The inner surface 14 of the cover substrate 12, the inner surface 17 of the base substrate 15 and the peripheral spacer 16 define a chamber 19. The substrates are typically made from glass, however, other materials such as polymers, polystyrene, fused silica, polypropylene, metal and combinations thereof can be used.

Preferably, the chamber 19 has a height less than 50 microns and a fluid volume of less than about 75 microliters. In certain embodiments, the height of the spacer and the corresponding height of the chamber are about 10 microns and the fluid volume of the chamber is as low as about 15 microliters. Of course, a person skilled in the art will understand that the exact volume of the chamber will depend on the height of the chamber and the length and width dimension of the chamber.

When the peripheral spacer is greater than about 30 microns in height, the spacer can be made from glass frit. Glass frit can be screen printed around the periphery of the cover slide and then fired at about 550° C. The seal then can be polished prior to sealing to the base substrate. When the peripheral spacer is less than about 30 microns in height, and particularly when the spacer is about 10 microns in height, better results have been obtained by using a polymer such as, for example, a plastic film to manufacture the peripheral spacer.

Still referring to FIGS. 1-2, fluid ports 20, 22 are disposed on opposite ends of the specimen area and extend through the cover substrate 12. The ports are in fluid communication with reservoirs 24 and 26 capable of holding a volume of fluid. In preferred embodiments, each reservoir 24 and 26 is capable of holding a volume of fluid equal to at least one-half of the volume of the chamber 19. In FIGS. 1 and 2, the reservoirs 24 and 26 are shown as two capillary tubes that protrude in a generally perpendicular direction from the cover substrate 12. It will be appreciated that other configurations can be used and still be within the scope of the invention. For example, the reservoirs can be funnel-shaped, allowing the height of the reservoirs to be reduced. In an alternative embodiment described in more detail below, the reservoir comprise channels running parallel to the surface of the substrates.

The hybridization device further includes apparatus for moving fluid through the reservoirs 24, 26, the ports 20, 22 and the chamber 19. For example, a pumping device 30 connected to at least one of the ports 22 by tubing or any suitable connector 32 can move the fluid through reservoir 24, port 22, chamber 19, port 20 and port 26 in an oscillating motion to provide mixing of the fluid in the chamber 19. For chambers having a height between about 30 and 50 microns, a syringe pump is sufficient to move the fluid back and forth through the chamber 19. For chambers having heights lower than about 30 microns, applicants have discovered that a pressure and vacuum source connected to at least one of the ports 20 moves fluid through the apparatus and provides movement and mixing of the fluid.

In certain embodiments, a sensor 34 can be placed proximate to at least one of the reservoirs 24 to monitor the fluid volume therein. As shown in FIG. 1, a sensor 34 is mounted on sidewall of the reservoir 24. The sensor is in electrical communication with the pump 30. A controller 36, such as a computer or other suitable control device can control the pump operation based on the fluid volume in the reservoir 24. For example, in embodiments in which the fluid movement is provided by a pressure and vacuum device, when the fluid volume is such that the fluid level is below the sensor 34, vacuum pressure will draw fluid upwardly through reservoir 24 and the fluid in reservoir 26 will move downwardly and through the chamber 19. When the fluid reaches the level of the sensor 34 in the reservoir 24, the sensor signals the controller, and the controller signals the pump 30 to switch to pressure to push fluid downwardly through the reservoir 24, through port 22, chamber 19, port 20 and upwardly through reservoir 26. During a biochemical reaction experiment such as a hybridization reaction, this system can be used to oscillate fluid in a back and forth motion through the chamber to maintain fluid movement and mixing during hybridization.

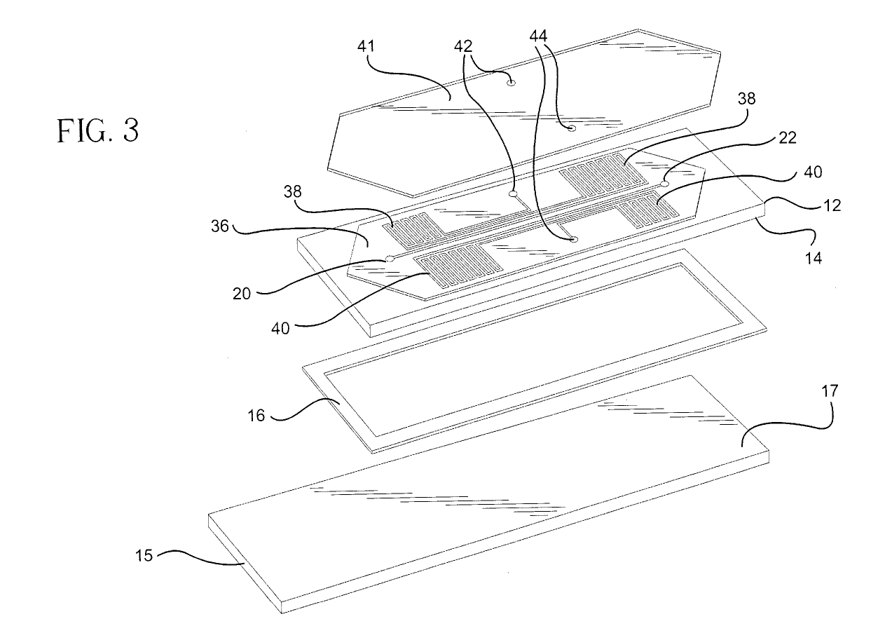

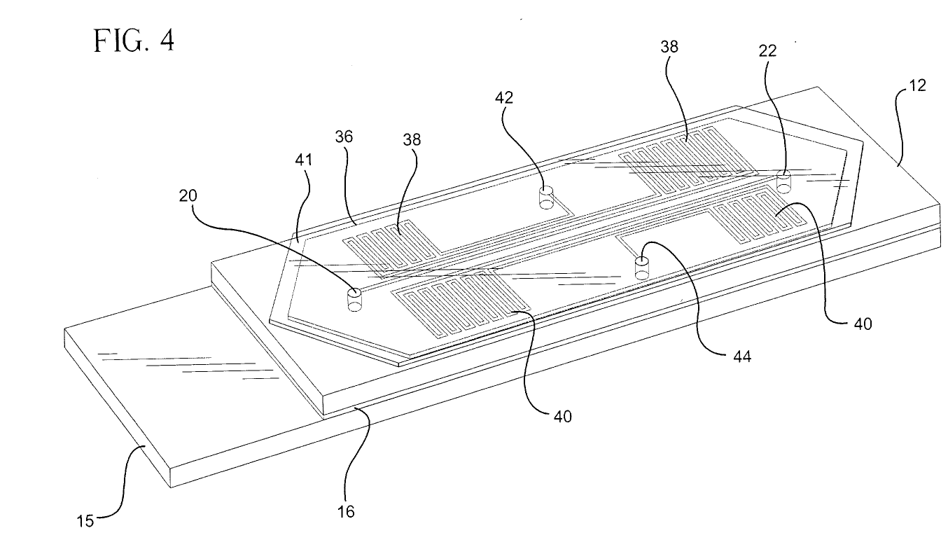

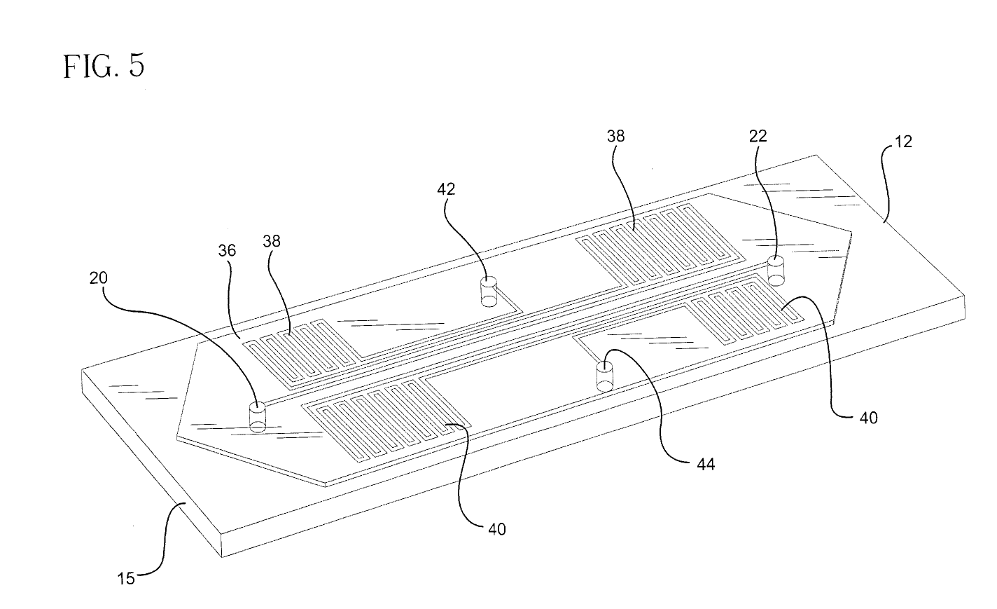

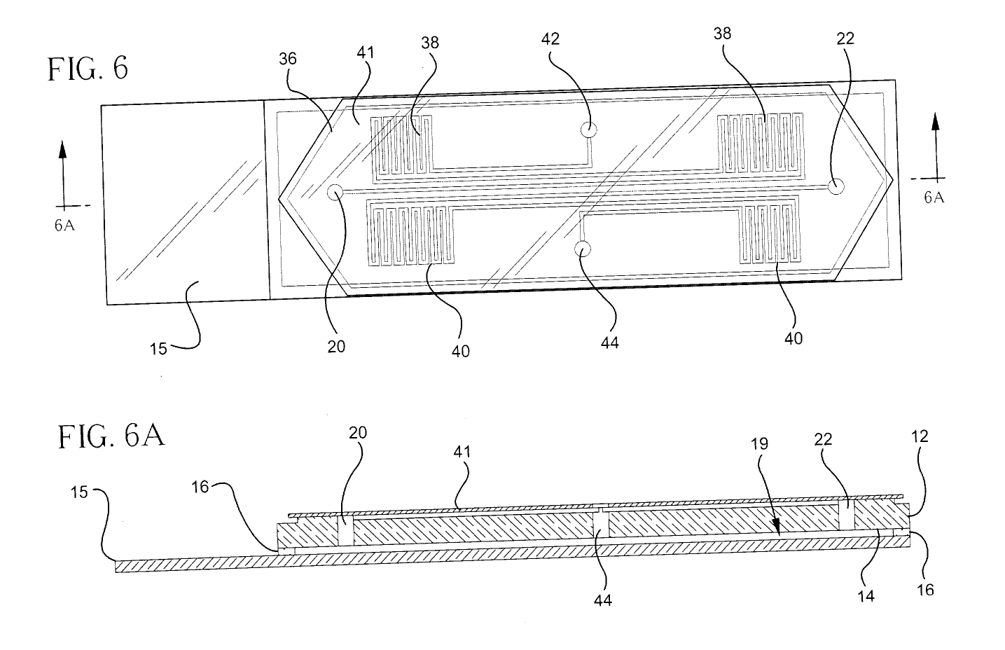

FIGS. 3-6A show an alternative embodiment of the present invention. As in the previously described embodiment, a cover substrate 12 having an inner surface 14 and a peripheral spacer 16 is attached in a substantially parallel configuration with a base substrate 15 having an inner surface 17. The spacer 16, and the inner surfaces 14 and 17 define a chamber 19. In this embodiment, the height of the spacer 16 is about 10 microns and the spacer can be made from a polymer such as a polymeric film. Cover substrate 12 includes ports 20 and 22 protruding therethrough. The ports 20 and 22 are in fluid communication with channels formed on an upper surface 36 of the cover substrate 12. Port 20 is in fluid communication with fluid channel 38, and port 22 is in fluid communication with fluid channel 40. The channels 38 and 40 are enclosed by a third substrate 41. Fluid and pump connections 42 and 44 extend through the third substrate 41 to permit fluids to be supplied to the channels 38 and 40, which are in fluid communication with the chamber 19 via ports 20 and 22.

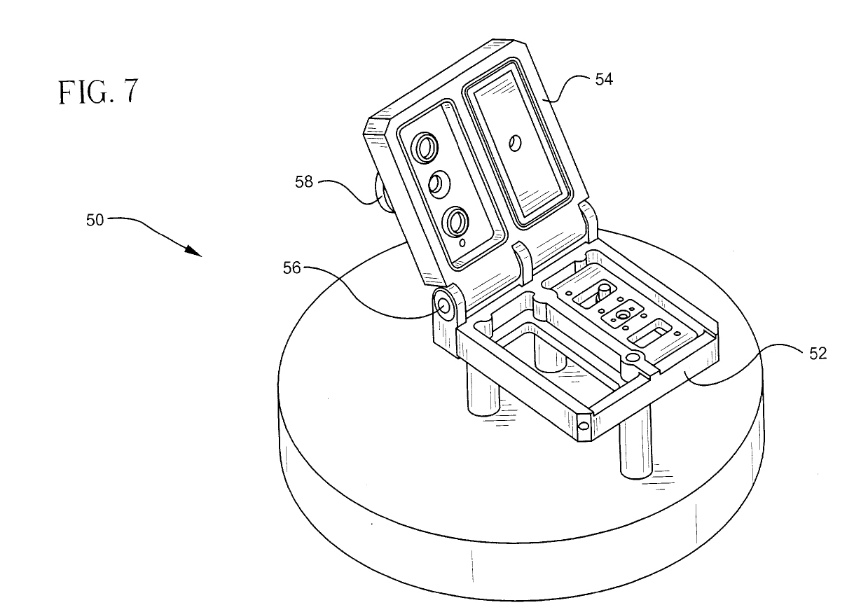

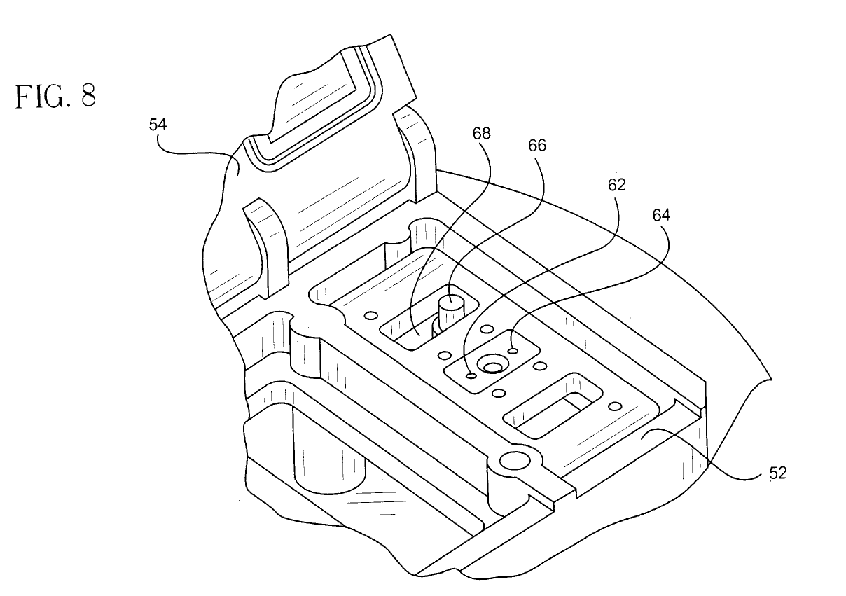

Referring now to FIG. 7, the reaction chamber device shown in FIGS. 3-6A can be used with a biochemical reaction station 50 of the type shown in FIG. 7. The reaction station 50 includes a reaction chamber device holder 52 and a lid 54 that opens and closes by hinge 56. The lid 54 can be securely closed tight by tightening screw 58. Referring to FIG. 8, which shows the reaction chamber device holder 52 in closer detail, fluidic or pumping connections 62 and 64 are located such that they align with and connect to fluid and pump connections 42 and 44 of the hybridization device. A sensor 66 is located in a window 68 that monitors the fluid level in one of the channels 38 or 40.

In use, the reaction chamber device shown in FIGS. 3-6A can be placed in a reaction chamber station of the type shown in FIGS. 7 and 8 and positioned such that connections 42 and 44 are aligned with connections 62 and 64 of the reaction chamber station. Molecules can be affixed or bound on the surface of the base substrate 16, and fluid containing analyte molecules can be introduced through connections 62 and 64 and pumped into channels 38 and 40 until the chamber 19 and one of the channels 38 or 40 is filled with fluid. Preferably, the volume of the channels 38 and 40 is at least equivalent to the volume of the chamber. After the chamber 19 and one of the channels is filled with fluid, a pressure and vacuum source for fluid movement (not shown) can be connected to the connections 62 and 64. In a manner similar to the embodiment described above in FIG. 1, sensor 66 can be connected to a controller which is in turn connected to the fluid movement device (not shown). The sensor sends signals to the controller, which in turn sends a signal to the fluid movement device to operate in either the pressure or vacuum mode. In this manner, the fluid movement device can move fluid back and forth through the chamber 19 to maintain fluid movement during a hybridization experiment.

Without intending to limit the invention in any manner, the invention will be more fully understood and described by the following examples, in which the conventional cover slip hybridization method was compared to certain embodiments of hybridization devices including spacers having heights of 100 microns, 50 microns and 10 microns.

### EXAMPLES

Preparation of Target Solutions and Immobilized Probes

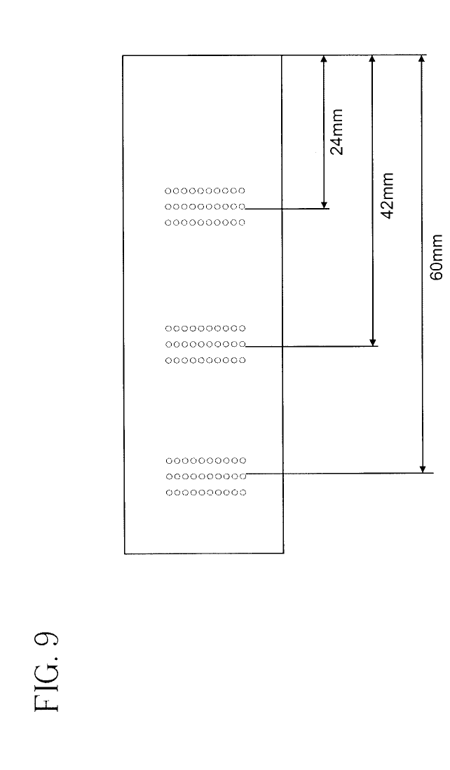

Double stranded DNA of each human gene in Table 1 were first amplified by the polymerase chain reaction (PCR). PCR products were then purified with the Qiagen PCR purification column (Qiagen, Inc., Valencia, Calif.). Purified PCR products of CASP7, CHES1, CYP4F2, CYP4F3, CYP24, RAQ, TNFRSF6, USP5, USP14, and USP15 genes were separately used as a template for printing onto Corning CMT-GAPS™ slides in the pattern shown in FIG. 9 or used as template to prepare Cy3 and Cy5 probes. Each product of labeling reaction was mixed in different ratio according to Table 2. Hybridization was done with an equal amount of Cy3 probe for each gene. The equal amount of target DNA (200 ng each for all 10 genes) was mixed and labeled with Cy3. Two ng of Cy3 probe was used for each hybridization (the concentration Cy3 probe of each gene is 200 pg/hyb for Ci). The total 2 ug of Cy3 labeled DNA was enough for 1000 hybridizations.

TABLE I

| Gene | | Size, | | Accesion |
|---|---|---|---|---|
| ID | Symbol | bp | UniGene ID | No. |
| 1 | CASP7 | 500 | Hs. 9216 | NM_001227 |
| 2 | CHES1 | 501 | Hs. 211773 | NM_005197 |
| 3 | CYP4F2 | 492 | Hs. 101 | NM_001082 |
| 4 | CYP4F3 | 497 | Hs. 106242 | NM_000896 |
| 5 | CYP24 | 510 | Hs. 89663 | NM_000782 |
| 6 | RAQ | 496 | Hs. 13495 | NM_006268 |
| 7 | TNFRSF6 | 506 | Hs. 82359 | NM_000043 |
| 8 | USP5 | 503 | Hs. 3759 | NM_003481 |
| 9 | USP14 | 490 | Hs. 75981 | NM_005151 |
| 10 | USP15 | 500 | Hs. 23168 | AF106069 |

TABLE II

| | Con. of | Conc. of |
|---|---|---|
| | Cy3 probe | Cy5 probe |
| Gene | [pg/ul] | [pg/ul] |
| CHES1 | 200 | 100000 |
| TNFR | 200 | 10000 |
| CASP7 | 200 | 10000 |
| CYP4F2 | 200 | 1000 |
| USP5 | 200 | 100 |
| CYP4F3 | 200 | 100 |
| RAQ | 200 | 25 |
| USP14 | 200 | 25 |
| USP15 | 200 | 5 |
| CYP24 | 200 | 5 |

Labelling of Cy5 probe was done as follows: mix 10 ug Gene 1, 1 ug Gene 2 and Gene3, 100 ng Gene 4, 10 ng Gene 5 and Gene 6, 1 ng Gene 7, 0.1 ng Gene 8 and 9, 0.01 ng Gene10. The DNA mixture was labeled with Cy5, and 1/100 of the Cy5 probe was used for hybridization. The Cy5 probe concentration ranges from 100 ng/hyb to 1 pg/hyb for Ci.

Microarray Printing

To print the microarrays (immobilized targets), purified PCR product of each gene in Table 2 was prepared at 250 ng/μL in 50% DMSO and 0.25×SSC (Gen I ink), and the immobilized targets were arrayed (“spotted”) on Corning CMT-GAPS™ coated glass slides with the Cartesian Pixsys 5500C Arrayer (Cartesian Technologies, Irvine, Calif.). Three time three triplicates were used for each hybridization assay as shown in FIG. 9. Two Bacillus subtilis genes were used as a negative control. After spotting, the glass slide was heated at 80° C. for 4 hours. Then slides were stored in a dessicator.

### Preparation of Hybridization Buffer and PCR Amplification of 10 Human Genes and Three B. Subtilis Genes

In order to better evaluate the performance of the chamber, two negative controls were also included in this set of experiments. The final PCR products were from eight of 100 ul reactions for each gene. After amplification, the PCR products were precipitated with 1× v isopropanol and dissolved in 200 ul of 10 mM Tris.Cl [pH 8]. They were further purified by Qiagen PCR purification columns and eluted with 50 ul of nuclease free water. PCR products were checked on 1% agarose gel, and DNA concentrations were measured with spectrophotometer. DNA concentration was adjusted to 500 ng/ul. Each gene had a final concentration of 250 ng/ul dsDNA in 50% DMSO/0.25×SSC.

| Cocktail in ul: | |
|---|---|
| dd water | 72.5 |
| 10× Buffer | 10 |
| 25 mM MgCl2 | 6 |
| 10 mM dATP | 2 |
| 10 mM dTTP | 2 |
| 10 mM dCTP | 2 |
| 10 mM dGTP | 2 |
| 5 u/ul Tag [Promega] | 0.5 |
| 10 ng/ul DNA template | 1 |
| 20 uM Primer pair | 2 |

PCR parameters:

| | |
|---|---|
| Step 1: | 95 C., 2 min |
| Step 2: | 94 C., 30 sec |
| Step 3: | 60 C., 30 sec |
| Step 4: | 72 C., 30 sec |
| Step 5: | Go to step 2 for 29 times |
| Step 6: | 72 C., 5 min |
| Step 7: | 4 C. for ever |

Labelling Probes

For Cy3 labeling, each gene had 200 ng of double stranded DNA (dsDNA). For Cy5 labeling, different amounts of dsDNA were used for different genes. See Table III for details.

Labeling reaction in ul:

| | |
|---|---|
| Cy3 | Cy5 |

| Dd water | 4 | | 17 | |
|---|---|---|---|---|
| DNA mix | 4 | [2 ug] | 31 | [12.1 ug] |
| 3 ug/ul Random hexamer | 1 | | 6 | |
| | 95 | C., 5 min | | |
| 10 × buffer | 2 | | 12 | |
| 10/1 mM dATG/C | 2 | | 12 | |
| 1 mM Cy3 dCTP | 2 | | 1 | mM Cy5 |
| | | | | dCTP 12 |
| 0.1 M DTT | 1 | | 6 | |
| 5 u/ul Klenow fragment | 4 | | 24 | |
| | 60 | ul per reaction × 2 | | |
| | 37 | C., 2 hours | | |
| | 95 | C., 3 min | | |

cool on ice

Purified by Qiagen PCR purification column

| | |
|---|---|
| Measure: | A260/280/550 for Cy3 probe |
| | A260/280/650 for Cy5 probe |

One ul of Cy3 and/or Cy5 probe per 60 ul of hybridization buffer.

TABLE III
Probe Concentration calculated based
on template DNAs added for labeling

| | Con. of | Conc. Of |
|---|---|---|
| | Cy3 Probe | Cy5 probe |
| Gene | [pg/ul] | [pg/ul] |
| CHES1 | 200 | 100000 |
| TNFR | 200 | 10000 |
| CASP7 | 200 | 10000 |
| CYP4F2 | 200 | 1000 |
| USP5 | 200 | 100 |
| CYP4F3 | 200 | 100 |
| RAQ | 200 | 25 |
| USP14 | 200 | 25 |
| USP15 | 200 | 5 |
| CYP24 | 200 | 5 |

Hybridization and Analysis

A total volume of 20 to 150 μL of multiplex hybridization assay mixture was used for each hybridization assay. The multiplex hybridization assay mixture contained both Cy3 and Cy5 labeled probes of each gene (Table 2) and hybridization buffer. The prehybridization and the washing steps were performed in a coplin jar in a conventional way. To study the hybridization step independently from the other part of the process (prehybridization or washing step), the hybridization chamber was used only for the hybridization step. For the conventional cover slip method, seventy-five microliters of the same multiplex hybridization assay mixture (mobilized targets) used for the hybridization chamber were pipetted onto the surface of the arrayed and pre-hybridized Corning GAPS™ coated glass slide. A glass cover slip was placed on top of the mixture and it was placed inside a custom-made humidity chamber. To start the array hybridization, the chamber was placed into a 42° C. water bath. After hybridization, the glass slides were washed, dried and scanned with the GenePix 4000A Microarray Scanner. Data were analyzed with the GenePix Pro 3.0 software (Axon Instruments, Inc., Foster City, Calif.).

Assembly of Hybridization Chambers

FIGS. 1 and 2 show the embodiment of the hybridization chamber that was used for the biological validation. The thickness of the peripheral spacer controls the total volume required in the hybridization chamber. Two kinds were used. Spacers around 50 microns and above were made of fritted glass. 200 microns thick glass paste was deposited by screen-printing. The footprint of 8 chambers was done in the same manufacturing batch. The paste was fritted at 550° C. The 8 chambers were then diced and polished individually to the required height of 100 or 50 microns. Initial experimentation showed that for spacers below about 50 microns, the pressure inside the chamber to move the fluid was too large for the frit to make a good seal. Accordingly, polymer seals were engineered with polymeric film. The stretched film was deposited over the glass body heated to 80° C. for a few seconds, cooled down and cut to the required design to provide a peripheral spacer. The thickness of the film was measured between 10 and 15 microns. Proper sealing under movement was demonstrated up to a temperature of 60° C.

Fluid Movevement in Chambers

Fluid movement was accomplished with a syringe pump from World Precision Instrument in hybridization chambers with a gap of 50 microns and above. For gaps below 30 microns, a source of compressed air and a vacuum were used to build a pressure/vacuum with electronically controlled valves. Generally, the pressure and vacuum connections were connected to only one of the ports or channels. For hybridization chambers having fluid gaps or spacers as low as 10 microns, vacuum and pressure of about 400 mbar was used to push and pull the fluid through the system. For larger fluid gaps or spacers, the pressure and vacuum could be decreased to as low as about 100 mbar or lower, depending on the volume of the chamber, the size of the channels and the ports. Capacitor detectors were used to detect the fluid level in the capillary to determine when to switch between vacuum and pressure. The system was set up to work with one or both capillaries. After tuning, a significant volume of fluid (approximately a third of the hybridization chamber volume) could be moved back and forth overnight in a 10-micron chamber without a significant drift.

Results Comparing Chambers Having Gaps Between 100 Microns and 10 Microns

The following set of experiments demonstrates the effect of probe amount by weight on hybridization signal. Hybridization chambers having lower height between the inner surfaces of the cover substrate and the base substrate (referred to as the gap) have smaller volumes, which allow for higher concentration. The hybridization mixture was used at an amount referred to as 1× and diluted 10 or 50 times which is referred to as 0.1× or 0.02×.

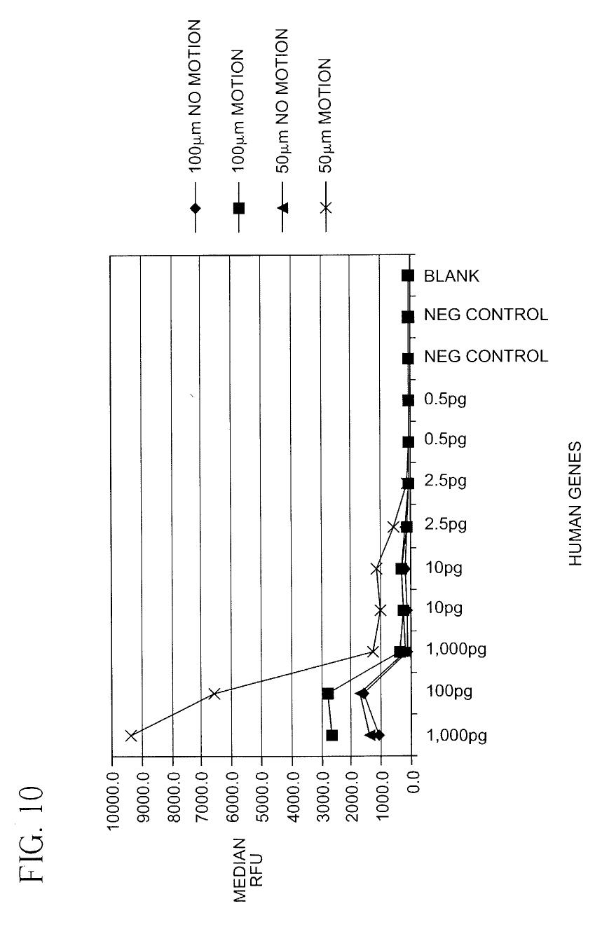

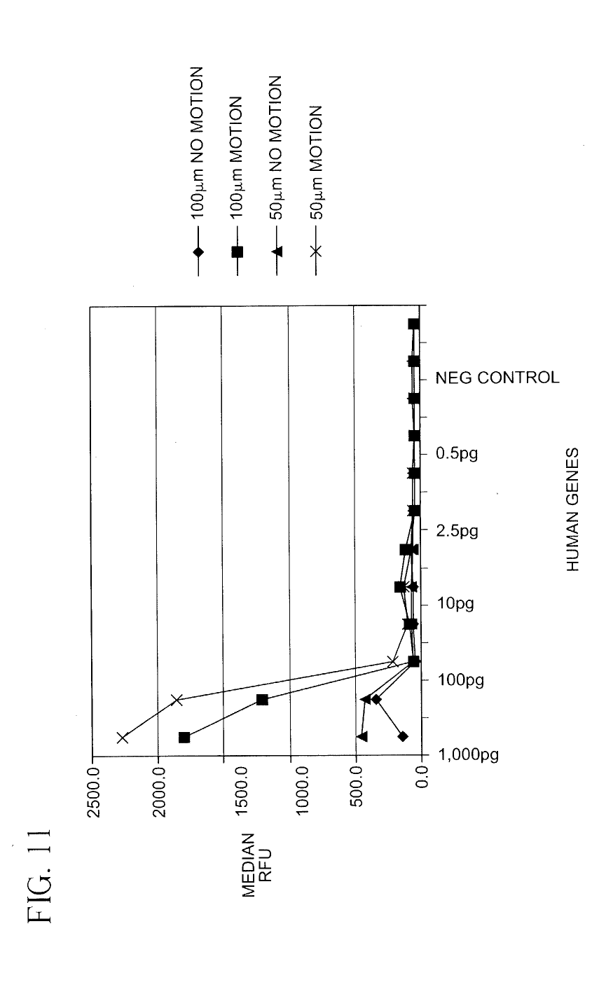

First, hybridization in 100 and 50-micron gap hybridization chambers were compared. FIG. 10 shows that hybridization with lx probe together with movement of the fluid in a 50 or 100 micron gap chamber, RFU signals are 2 to 5 times higher than in the same hybridization without any movement. The higher concentration range leads to a larger difference between chambers using fluid movement and chambers not using fluid movement. Another set of experiments was performed at a probe concentration of 0.1×. FIG. 11 shows that by moving the fluid there is a significant gain in RFU level specially for the genes in larger abundance.

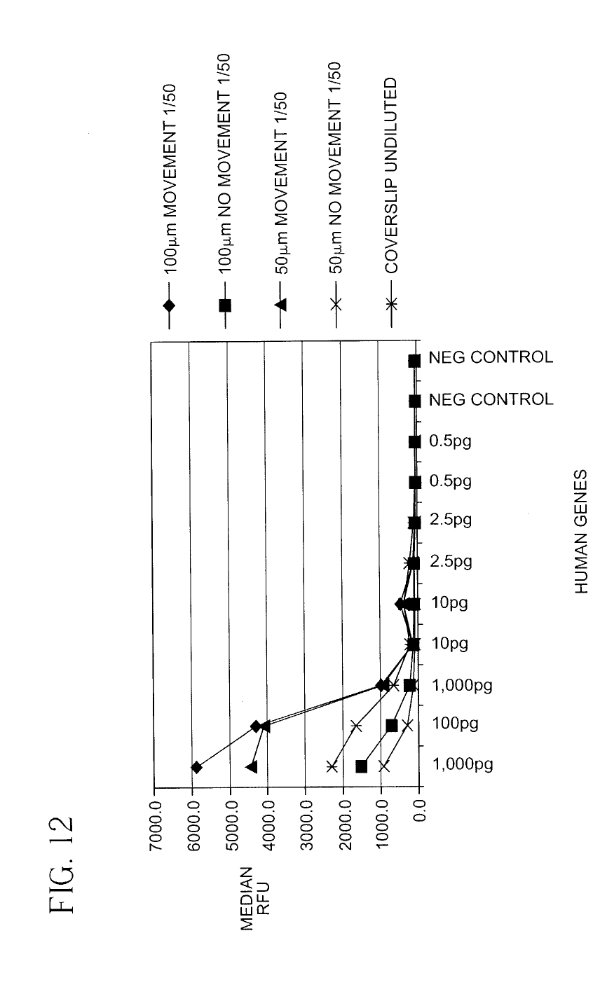

The next set of data compare a 10 um-gap hybridization chamber with a 0.02× probe versus a 50 μm-gap chamber at 1× probe with and without movement and a cover slip hybridization. FIG. 12 shows that the probe diluted 50-fold in a 10 micron chamber gives signal intensities equivalent to or higher than the same probe at 1× amount in a 50 micron gap chamber. The same hybridization signals are up to 3 times higher than the cover slip hybridization at 1× of probe.

Next, the sensitivity gain in a hybridization chamber having a 10 micron gap and fluid movement via fluid movement using vacuum and pressure was compared with a conventional cover slip method. Table 4 shows the results of experiment where hybridization in a chamber having a height of 10-microns and fluid movement via pressure and vacuum with a probe diluted 50 times is compared to the hybridization under a cover slip. The results show again that by using smaller volume and moving the fluid, the signal intensities of the diluted probe in the hybridization chamber are higher than the same signal from non-diluted probe under the cover slip.

TABLE IV

| | COVER SLIP | 10 MICRON |
|---|---|---|
| GENE | PROBE 1× | PROBE 0.02× |
| CASP7 | 46 | 142 |
| CYP4F2 | 33 | 104 |
| CYP4F3 | 10 | 29 |
| RAQ | 13 | 23 |

Over 200 hybridization experiments have been run using the cover slip method utilizing a fluid volume of 75 microliters and compared with a hybridization chamber having a gap of 100 microns and fluid movement via syringe pump, a hybridization chamber having a 50 micron gap having a fluid volume of 75 microliters with fluid movement via a syringe pump and a hybridization chamber having a 10 micron gap and a fluid volume of 20 microliters with fluid movement via pressure and vacuum. These experiments showed that the hybridization chamber having a having a 100 micron gap showed a gain in sensitivy compared to the cover slip method of less than 5 time. The 50 micron gap hybridization chamber showed a 3-10 gain in sensitivity compared to the cover slip method, and the hybridization chamber with a 10 micron gap showed a sensitivity gain greater 50 compared to the cover slip method.

The experiments above demonstrate that by decreasing the volume from 150 to 20 microliters and by providing movement in the hybridization with a pumping device, the hybridization signal can be increased significantly. The gain in sensitivity is driven by two factors, reducing the volume of hybridization which allow for higher concentration and forcing the fluid through the chamber which increases the chance for a probe in solution to meet its counter part on the surface. Providing fluid movement and mixing in a small volume chamber reduced the dependence of the reaction on diffusion.

It will be apparent to those skilled in the art that various modifications and variations can be made to the present invention without departing from the spirit or scope of the invention. Thus, it is intended that the present invention cover modifications and variations of this invention provided they come within the scope of the appended claims and their equivalents.

What is claimed is:

1. A biochemical reaction device comprising:
a generally planar base substrate having an inner surface including a specimen area having at least one biomolecule attached thereto;
a generally planar cover substrate having an inner surface opposing and substantially parallel to the inner surface of the base substrate;
a peripheral spacer disposed between the inner surfaces of the base substrate and the cover substrate and forming a fluid tight seal therebetween, wherein the inner surfaces of the base substrate and the peripheral spacer define a chamber having a height less than 50 microns and a fluid volume of less than 75 microliters;
at least a pair of fluid ports disposed on opposite ends of the specimen area, each of the ports in fluid communication with channels capable of holding fluid; and
means for moving fluid through the channels, the pair of fluid ports and the chamber.

2. The device of claim 1, wherein the peripheral spacer is made from a glass frit.

3. The device of claim 1, wherein the peripheral spacer is made from a polymer.

4. The device of claim 1, wherein the spacer is made from a material selected from the group consisting of glass, polymers, a liquid and combinations thereof.

5. The device of claim 1, wherein the height of the chamber is less than 20 microns and the fluid volume of the chamber is less than 30 microliters.

6. The device of claim 1, further including fluid channels disposed between a third generally planar substrate and an upper surface of the cover substrate and the direction of the fluid flow through the channels is generally parallel to the inner surface of the cover substrate.

7. The device of claim 1, wherein the means for moving the fluid includes pressure and vacuum connected to at least one of the pair of ports.

8. The device of claim 1, wherein the means for moving fluid includes a syringe pump connected to at least one of the pair of ports.

9. The device of claim 6, wherein a sensor is disposed proximate at least one of the channels to monitor the volume of fluid in the channel.

10. The device of claim 9, wherein the sensor is in communication with the pressure and vacuum and the sensor is operative to control the pressure and vacuum to change the direction of fluid flow.

11. A biomolecular reaction device comprising:
a generally planar base substrate having an inner surface including a specimen area containing at least one biomolecule;
a generally planar cover substrate having an inner surface opposing and substantially parallel to the inner surface of the base substrate;
a peripheral spacer disposed between the inner surfaces of the base substrate and the cover substrate and forming a fluid tight seal therebetween, wherein the inner surfaces of the base substrate and the peripheral spacer define a chamber having a height less than 50 microns and a fluid volume of less than 75 microliters;
at least a pair of fluid ports disposed on opposite ends of the specimen area, each of the ports in fluid communication with the specimen area and a reservoir for holding fluid; and
a fluid movement device in fluid communication with at least one of the reservoirs for moving fluid between the pair of reservoirs and the specimen area.

12. The device of claim 11, wherein each reservoir includes a series of fluid channels disposed above an upper surface of the cover substrate.

13. The device of claim 11, wherein each reservoir includes a series of fluid channels disposed between an upper surface of the cover substrate and a third substrate arranged substantially parallel to the cover substrate.

14. The device according to claim 11, wherein the fluid movement device includes a source of pressure and vacuum connected to the pair of reservoirs.

15. The device according to claim 14, further including a sensor operative to monitor the fluid volume in the reservoirs and in electrical communication with the vacuum and pressure to control the direction of fluid flow by alternating between operation of the vacuum and operation of the pressure.

16. A method of performing a hybridization assay comprising:
providing a hybridization device including a generally planar base substrate having an inner surface including a specimen area having an array of biomolecules attached thereto, a generally planar cover substrate having an inner surface opposing and substantially parallel to the inner surface of the base substrate, a peripheral spacer disposed between the inner surfaces of the base substrate and the cover substrate and forming a fluid tight seal therebetween, wherein the inner surfaces of the base substrate and the peripheral spacer define a chamber having a height less than 50 microns and a fluid volume of less than 75 microliters, and at least a pair of fluid ports disposed on opposite ends of the specimen area, each of the ports in fluid communication with reservoirs capable of holding volume of fluid at least equal to the fluid volume in chamber; and
moving fluid between the pair of reservoirs and their respective ports and through the chamber.

17. The method of claim 16, wherein the microarray includes probe molecules and the fluid contains a mixture of biomolecules, at least some of which are complementary target biomolecules.

18. The method of claim 17, wherein the fluid is moved through the pair of reservoirs by a pump connected at least one of the pair of reservoirs.

19. The method of claim 18, wherein the pump includes a syringe pump.

20. The method of claim 16, wherein the fluid is moved with a pressure and vacuum connected to at least one of the reservoirs.

21. The method of claim 18, wherein the pump includes pressurized gas for displacing the fluid.

22. The method of claim 18, wherein the pump includes piezolelectric pump for displacing the fluid.

23. The method of claim 18, further comprising the step of monitoring the volume of fluid in at least one of the reservoirs.

24. The method of claim 23, further comprising the step of controlling operation of the pump based on the volume of fluid in at least one of the reservoirs.

25. The method of claim 24, further comprising controlling the direction of fluid movement in the chamber based on the volume of fluid in at least one of the reservoirs.
